(19) United States
(12) Patent Application Publication
(10) Pub. No.: US 2024/0189043 A1
(43) Pub. Date: Jun. 13, 2024

(54) SURGICAL NAVIGATION SYSTEM AND METHOD, AND ELECTRONIC DEVICE AND READABLE STORAGE MEDIUM

(71) Applicant: Jedicare Medical Co., Ltd., Shanghai (CN)

(72) Inventors: Fei Sun, Shanghai (CN); Yi Zhu, Shanghai (CN); Xiaojie Guo, Shanghai (CN); Fuli Cui, Shanghai (CN); Ying Shan, Shanghai (CN)

(21) Appl. No.: 18/552,077

(22) PCT Filed: Mar. 18, 2022
(86) PCT No.: PCT/CN2022/081728

§ 371 Date: Sep. 22, 2023

(30) Foreign Application Priority Data
Apr. 1, 2021 (CN) 202110358153.3

(51) Int. Cl. A61B 34/20; G06T 7/246; G06T 7/73; G06V 10/40; G06V 10/74; G06V 20/50

(52) U.S. Cl. CPC A61B 34/20; G06T 7/246; G06T 7/73; G06V 10/40; G06V 10/761; G06V 20/50; A61B 2034/2055; A61B 2034/2065; G06T 2207/30004; G06T 2207/30241; G06V 2201/03

(57) ABSTRACT

The present solution provides a surgical navigation system and method, and an electronic device and a readable storage medium. The surgical navigation system comprises: an image obtaining module, for obtaining a surgical scene image; an image recognition module, for executing image recognition on the surgical scene image to obtain a first recognition result, wherein the first recognition result is used to represent an identifier contained in the surgical scene image; an instruction obtaining module, for, based on the first identification result, obtaining a corresponding interaction instruction; an instruction execution module, for, based on the interaction instruction, controlling the surgical navigation system to execute a corresponding surgical navigation step. The implementation of the technical solutions of the present application can reduce the chance of misjudgment when the surgical navigation system is controlled.

### TECHNICAL FIELD

The present application relates to medical field and, in particular, to a surgical navigation system and method, electronic device and readable storage medium.

### BACKGROUND

A surgical navigation system is used for accurately correlating a patient's pre-operative or intra-operative image data with the patient's anatomical structure on an operating table, and tracking a surgical device, displaying and updating the positions of the surgical device on the patient's image in the form of a virtual probe in real time during the surgical procedure. So surgeons are able to see at a glance the positions of the surgical device relative to the patient's anatomical structure, and it can make the surgical procedure faster, more precise and safer.

Augmented reality devices can significantly improve the efficiency of wearers' work, and they can be used for realizing human-machine interaction mainly in the manner of gestures and voices, and the like. When the augmented reality devices mentioned above are applied to the surgical navigation system, they have the following shortcomings. If gestures are used to realize the human-machine interaction with the surgical navigation system, misjudgments may occur due to blood contamination of the surgeons' gloves or the appearance of more than one hand within the camera's field of view, and the like. If voices are used to realize the human-machine interaction with the surgical navigation system, false triggering may be caused due to necessary intra-operative communications.

### SUMMARY

In order to address at least one of the aforesaid technical problems, the present application provides a surgical navigation system, method, electronic device, and readable storage medium.

According to the first aspect of the present application, there is provided a surgical navigation system, comprising:
an image obtaining module, for obtaining a surgical scene image;
an image recognition module, for executing image recognition on the surgical scene image to obtain a first recognition result, wherein the first recognition result is used to represent an identifier contained in the surgical scene image;
an instruction obtaining module, for, based on the first identification result, obtaining a corresponding interaction instruction;
an instruction execution module, for, based on the interaction instruction, controlling the surgical navigation system to execute a corresponding surgical navigation step.

Optionally, when the image recognition module is used for executing the image recognition on the surgical scene image to obtain the first recognition result, it is specifically used for:
extracting an image feature of the surgical scene image;
determining the first recognition result based on a similarity between the image feature of the surgical scene image and an image feature of the identifier.

Optionally, when the instruction obtaining module is used for, based on the first recognition result, obtaining the corresponding interaction instruction, it is specifically used for:
obtaining a surgical navigation stage at which the surgical navigation system is;
based on the first recognition result and the surgical navigation stage, obtaining the corresponding interaction instruction.

Optionally, when the instruction obtaining module is used for, based on the first recognition result, obtaining the corresponding interaction instruction, it is specifically used for:
obtaining a second recognition result by executing image recognition on the surgical scene image, wherein the second recognition result is used to represent a relative position of the identifier in a preset space, or represent a relative distance between the identifier and a preset target;
based on the first recognition result and the second recognition result, obtaining the corresponding interaction instruction.

Optionally, when the instruction obtaining module is used for, based on the first recognition result, obtaining the corresponding interaction instruction, it is specifically used for:
obtaining a third recognition result by executing image recognition on the surgical scene image, wherein the third recognition result is used to represent an orientation and/or angle of the identifier;
based on the first recognition result and the third recognition result, obtaining the corresponding interaction instruction.

Optionally, when the instruction obtaining module is used for, based on the first recognition result, obtaining the corresponding interaction instruction, it is specifically used for:
obtaining a fourth recognition result by executing image recognition on the surgical scene image, wherein the fourth recognition result is used to represent a degree to which the identifier is obscured;
based on the first recognition result and the fourth recognition result, obtaining the corresponding interaction instruction.

Optionally, when the instruction obtaining module is used for, based on the first recognition result, obtaining the corresponding interaction instruction, it is specifically used for:
obtaining a fifth recognition result by executing image recognition on the surgical scene image, wherein the fifth recognition result is used to represent an absolute motion trajectory or a relative motion trajectory of the identifier, wherein the absolute motion trajectory is a motion trajectory of the identifier relative to a stationary object, and the relative motion trajectory is a motion trajectory of the identifier relative to a set person;
based on the first recognition result and the fifth recognition result, obtaining the corresponding interaction instruction.

According to the second aspect of the present application, there is provided an information interaction method for a surgical navigation system, comprising:
obtaining a surgical scene image;
executing image recognition on the surgical scene image to obtain a first recognition result, wherein the first recognition result is used to represent an identifier contained in the surgical scene image obtained by the recognition;
based on the first recognition result, obtaining a corresponding interaction instruction.

Optionally, said executing image recognition on the surgical scene image to obtain the first recognition result comprises:
extracting an image feature of the surgical scene image;
obtaining the first recognition result based on the image feature of the surgical scene image and an image feature of the identifier.

Optionally, based on the first recognition result, said obtaining the corresponding interaction instruction comprises:
obtaining information on a surgical stage;
based on the first recognition result and the information on the surgical stage, obtaining the corresponding interaction instruction.

Optionally, based on the first recognition result, said obtaining the corresponding interaction instruction comprises:
obtaining a second recognition result by executing image recognition on the surgical scene image, wherein the second recognition result is used to represent a relative position of the identifier in a preset space, or represent a relative distance between the identifier and a preset target;
based on the first recognition result and the second recognition result, obtaining the corresponding interaction instruction.

Optionally, based on the first recognition result, said obtaining the corresponding interaction instruction comprises:
obtaining a third recognition result by executing image recognition on the surgical scene image, wherein the third recognition result is used to represent an orientation and/or angle of the identifier;
based on the first recognition result and the third recognition result, obtaining the corresponding interaction instruction.

Optionally, based on the first recognition result, said obtaining the corresponding interaction instruction comprises:
obtaining a fourth recognition result by executing image recognition on the surgical scene image, wherein the fourth recognition result is used to represent a degree to which the identifier is obscured;
based on the first recognition result and the fourth recognition result, obtaining the corresponding interaction instruction.

Optionally, based on the first recognition result, obtaining the corresponding interaction instruction comprises:
obtaining a fifth recognition result by executing image recognition on the surgical scene image, wherein the fifth recognition result is used to represent an absolute motion trajectory or a relative motion trajectory of the identifier, wherein the absolute motion trajectory is a motion trajectory of the identifier relative to a stationary object, and the relative motion trajectory is a motion trajectory of the identifier relative to a set person;
based on the first recognition result and the fifth recognition result, obtaining the corresponding interaction instruction.

According to the third aspect of the present application, there is provided an electronic device comprising a memory and a processor. The memory is used to store computer instructions. The computer instructions are executed by the processor to implement the method according to the second aspect of the present application.

According to the forth aspect of the present application, there is provided a readable storage medium with the computer instructions stored thereon. When the computer instructions are executed by a processor, the method according to the second aspect of the present application is implemented.

The following beneficial technical effects can be achieved by implementing the technical solutions of the present application. In the technical solutions of the present application, an identifier contained in a surgical scene image can be automatically recognized. Based on the identifier contained in the surgical scene image, a corresponding interaction instruction can be obtained, and then based on the interaction instruction, the surgical navigation system is controlled to execute a corresponding surgical navigation step. This enables an operator to control the surgical navigation system to execute the corresponding surgical navigation step by photographing the surgical scene with the identifier, without the need to use voices, gestures, and the like. Relative to the prior art, the implementation of the technical solutions of the present application can reduce the chance of misjudgment when the surgical navigation system is controlled.

### BRIEF DESCRIPTION OF THE DRAWINGS

The drawings illustrate exemplary embodiments of the present disclosure and are used to explain the principles of the present disclosure in conjunction with the descriptions thereof. The drawings are used to provide further understandings of the present disclosure, and they are included in and form a part of the Description.

FIG. 1 is a block diagram of a structure of a surgical navigation system according to an embodiment of the present application;

FIG. 2 is a schematic diagram of a surgical scene according to an embodiment of the present application;

FIG. 3 is a schematic diagram of a surgical scene according to another embodiment of the present application;

FIG. 4 is a schematic diagram of a surgical scene according to yet another embodiment of the present application;

FIG. 5 is a schematic diagram of a surgical scene according to yet another embodiment of the present application;

FIG. 6 is a schematic diagram of a surgical scene according to yet another embodiment of the present application;

FIG. 7 is a schematic diagram of a surgical scene according to yet another embodiment of the present application;

FIG. 8 is a flowchart of an information interaction method for a surgical navigation system according to an embodiment of the present application;

FIG. 9 is a block diagram of a structural of an electronic device according to an embodiment of the present application;

FIG. 10 is a schematic diagram of a structure of a computer system according to an embodiment of the present application.

### DETAILED DESCRIPTION OF THE EMBODIMENTS

The present application is described in further detail below in conjunction with the drawings and embodiments. It is understandable that the specific embodiments described herein are only for the purpose of explaining the relevant contents, rather than limiting the present application. In addition, it is to be noted that for easier description, only the portions relevant to the present application are shown in the drawings.

It is to be noted that the embodiments and the features in the embodiments of the present application may be combined with each other in case that there is no conflict. The present application will be described in detail below with reference to the drawings and in conjunction with the embodiments.

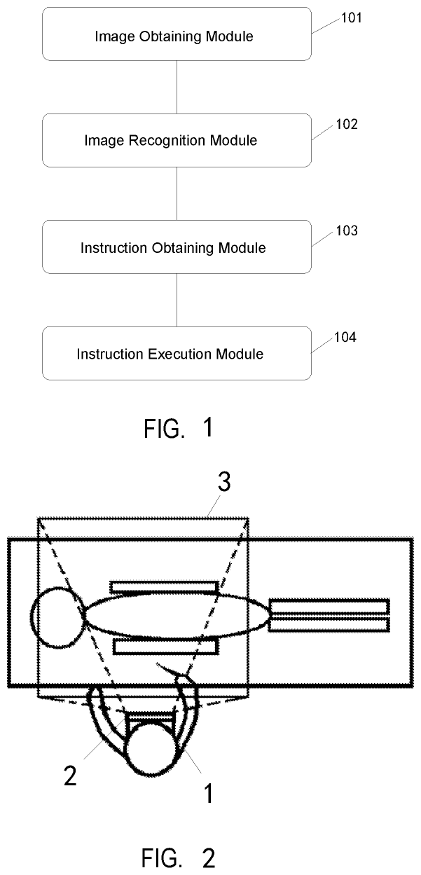

Referring to FIG. 1, a surgical navigation system, comprising:
an image obtaining module 101, for obtaining a surgical scene image;
an image recognition module 102, for executing image recognition on the surgical scene image to obtain a first recognition result, wherein the first recognition result is used to represent an identifier contained in the surgical scene image;
an instruction obtaining module 103, for, based on the first recognition result, obtaining a corresponding interaction instruction;
an instruction execution module 104, for, based on the interaction instruction, controlling the surgical navigation system to execute a corresponding surgical navigation step.

The surgical navigation system according to the embodiments of the present application may automatically recognize an identifier contained in a surgical scene image captured by a camera, and based on the identifier contained in the surgical scene image, obtain a corresponding interaction instruction, and then based on the interaction instruction, the surgical navigation system is controlled to execute a corresponding surgical navigation step. This enables an operator to control the surgical navigation system to execute the corresponding surgical navigation step by photographing the surgical scene with the identifier, without the need to use voices, gestures, and the like, so that the implementation of the technical solution of the present application can reduce the chance of misjudgment when the surgical navigation system is controlled. Additionally, the operation of the surgical navigation system is more convenient, so the impact on the operator's normal operations is reduced when he is operating the surgical system.

Wherein, it is known that the operator may photograph the surgical scene to capture the image thereof with a camera of a headwear device worn by him, referring to FIG. 2. FIG. 2 illustrates that an operator 1 captures a surgical scene image of a boxed area 3 by a camera of a headwear device 2 worn by the operator 1.

The identifiers in the embodiments of the present application may have at least one of the specific features from an optical feature, a pattern feature, and a geometric feature, to enable the images obtained by photographing the identifiers to have specific image features. For example, the identifier may be an information board, a planar positioning board, a two-dimensional code, and the like.

The surgical navigation system in the embodiments of the present application is triggered to execute a surgical navigation step corresponding to a specific identifier by recognizing the specific identifier. For example, the identifier may comprise a planar positioning board disposed on an operating table. When the planar positioning board is recognized, an interaction instruction for “triggering surgical area initialization is obtained, and a surgical navigation step for “surgical area initialization” is executed according to the interaction instruction. For example, the identifier may comprise a puncture handle. When the puncture handle is recognized, an interaction instruction for “triggering puncture navigation” is obtained, and a surgical navigation step for “puncture navigation” is executed according to the interaction instruction. For example, the identifier may comprise a two-dimensional code on the operating table. When the said two-dimension code is recognized, an interaction instruction for “triggering surgical navigation system to enter alignment” is obtained, and a surgical navigation step for “surgical navigation system alignment” is executed according to the interaction instruction.

Wherein, the surgical navigation steps may comprise a step for selecting a surgical device model. For example, the system pre-stores a library of surgical device models, including different types and versions of surgical device models, and the operator may point the camera of the headwear device at an identifier disposed on a surgical device (e.g., a two-dimensional code on the surgical device), by which a surgical device model is selected, so that the surgical device model of the navigation system is in conformity with the model of the real surgical application, and then the system proceeds to a next step for alignment. The surgical navigation steps may comprise a step for selecting a surgical navigation process, e.g., a plurality of identifiers may be disposed in a scene. Specifically, a first identifier (an information board) is disposed on an operating table and a second identifier (a two-dimensional code) is disposed on a surgical device. When the camera of the headwear device worn by the surgeon is facing the first identifier, a first stage (e.g., an alignment stage) is entered, and when the camera is facing the second identifier, another stage (e.g., a guided puncture stage) is entered. This is not limited by the embodiments of the present application.

Preferably, the identifiers in the embodiments of the present application are selected from recognizable patterns integral with a disposable surgical device, such as a two-dimensional code proposed on a puncture needle, so that the interacted identifiers can satisfy one of the two conditions of either repeated sterilization or disposable aseptic use.

The image recognition module in the embodiments of the present application may utilize existing image recognition algorithms for image recognition, such as blob detection algorithm, corner detection algorithm, and the like. Specifically, suitable algorithms may be selected according to the form of the identifiers, for example, where the identifier is a two-dimensional code proposed on the operating table or the surgical device, a corresponding two-dimensional code recognition algorithm may be directly adopted.

As an optional embodiment of the image recognition module, when the image recognition module is used to execute image recognition on the surgical scene image to obtain a first recognition result, it is specifically used for:
extracting an image feature of the surgical scene image;
determining the first recognition result based on a similarity between the image feature of the surgical scene image and an image feature of the identifier.

Specifically, the image recognition module is preset with a similarity threshold. When the similarity between the image feature of the surgical scene image and the image feature of the identifier is greater than the similarity threshold, it is determined that the surgical scene image contains the corresponding identifier.

Wherein, the image feature may include one or more features from a color feature, a texture feature, a shape feature and a spatial relationship feature.

As an optional embodiment of the instruction obtaining module, when the instruction obtaining module is used to, based on the first recognition result, obtain the corresponding interaction instruction, it is specifically used for:
obtaining a surgical navigation stage at which the surgical navigation system is;
based on the first recognition result and the surgical navigation stage, obtaining the corresponding interaction instruction.

In this embodiment, based on the first recognition result and the surgical navigation stage, the corresponding interaction instruction is obtained, so that the patterns corresponding to the same identifier may correspond to different interaction instructions at different surgical navigation stages, for the purpose of reducing the number of the identifiers; that is, when the image recognitions are executed on the surgical scene images, if it is recognized that the surgical scene images contain the patterns of the same identifier, but the surgical navigation stages of the surgical navigation system are different, the corresponding interaction instructions are different.

Taking the identifier being a two-dimensional code positioned next to the patient as an example, when the surgical navigation system is at the stage of not starting navigation, if it is recognized that the surgical scene image captured by the camera contains the two-dimensional code, an interaction instruction for “triggering the surgical navigation system to enter alignment stage” is generated; when the surgical navigation system is at the stage of alignment, if it is recognized that the surgical scene image captured by the camera contains the two-dimensional code, an interaction instruction for “re-alignment” is generated. During the specific application, when the camera is utilized to recognize the two-dimensional code next to the patient's body first time, it is triggered to initiate alignment for the scene; when an accident occurs in the process of the alignment which requires re-initiation of alignment, the entire process may be reset by recognizing the two-dimensional code at this position again only.

As an optional embodiment of the instruction obtaining module, when the instruction obtaining module is used to, based on the first recognition result, obtain the corresponding interaction instruction, it is specifically used for:
obtaining a second recognition result by executing image recognition on the surgical scene image, wherein the second recognition result is used to represent a relative position of the identifier in a preset space or represent a relative distance between the identifier and a preset target;
based on the first recognition result and the second recognition result, obtaining the corresponding interaction instruction.

In this embodiment, the preset space may be configured according to specific application needs, for example, the preset space may be configured to be a space corresponding to the surgical scene image. The preset target may be configured according to specific application needs, for example, the preset target may be configured to be a alignment point, a patient, and the like.

In this embodiment, different interaction instructions may be generated based on the identifier's positions, e.g., for the same process of reset of alignment, if the identifier is placed next to the patient, it is the entire process that should be reset, whereas if the identifier is placed near to a certain alignment point, it represents that only the alignment data for this position is reset.

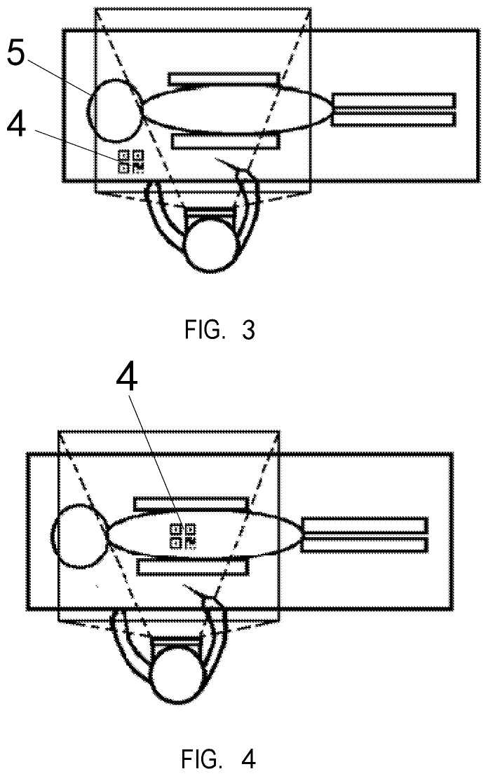

Taking the identifier being a two-dimensional code as an example, referring to FIGS. 3 and 4, the difference between FIGS. 3 and 4 is that the same identifier is at different positions, wherein the identifier 4 in FIG. 3 is positioned next to the patient, while the identifier 4 in FIG. 4 is proximity to the alignment point. The surgical scene image is captured for the area within the box in FIG. 3, the first recognition result obtained by executing image recognition on the surgical scene image is that the surgical scene image contains the identifier, and the second recognition result obtained by executing image recognition on the surgical scene image is that the relative distance between the two-dimensional code and the patient (specifically the patient's head) is less than a first preset distance threshold, and at this time, based on the first recognition result and the second recognition result, an interaction instruction for “triggering reset of entire process” is generated. The identifier in FIG. 4 is moved to be in proximity of the alignment point, and the surgical scene image is captured for the area within the box in FIG. 4, the first recognition result obtained by executing image recognition on the surgical scene image is that the surgical scene image contains the identifier, and the second recognition result obtained by executing image recognition on the surgical scene image is that the relative distance between the two-dimensional code and the alignment point is less than a second preset distance threshold, and at this time, based on the first recognition result and the second recognition result, an interaction instruction for “triggering reset of alignment data at current position only”. The first preset distance threshold and the second preset distance threshold may be configured to be 90%, and the like.

Specifically, in one embodiment, the relative distance between the identifier and the preset target is a relative distance between the extension line of the identifier and the preset target, for example, the relative distance between the extension line of a puncture needle and a rib. If the relative distance between the extension line of the puncture needle and the rib is less than a set value, it shows that there is a risk that the extension line of the puncture needle may touch the rib, and at this time, a corresponding interaction instruction for “triggering prompt message” is obtained to give a prompt, wherein the set value may be 0.

As an optional embodiment of the instruction obtaining module, when the instruction obtaining module is used to, based on the first recognition result, obtain the corresponding interaction instruction, it is specifically used for:
obtaining a third recognition result by executing image recognition on the surgical scene image, wherein the third recognition result is used to represent an orientation and/or angle of the identifier;
based on the first recognition result and the third recognition result, obtaining the corresponding interaction instruction.

In this embodiment, the orientation and/or angle of the identifier may be recognized with existing relevant algorithms, and the identifier has corresponding features so that the orientation and/or angle of the identifier can be obtained after the identifier is recognized on the image.

In this embodiment, by adjusting the orientation and/or angle of the identifier, the operator may trigger the corresponding interaction instruction to improve the convenience of control.

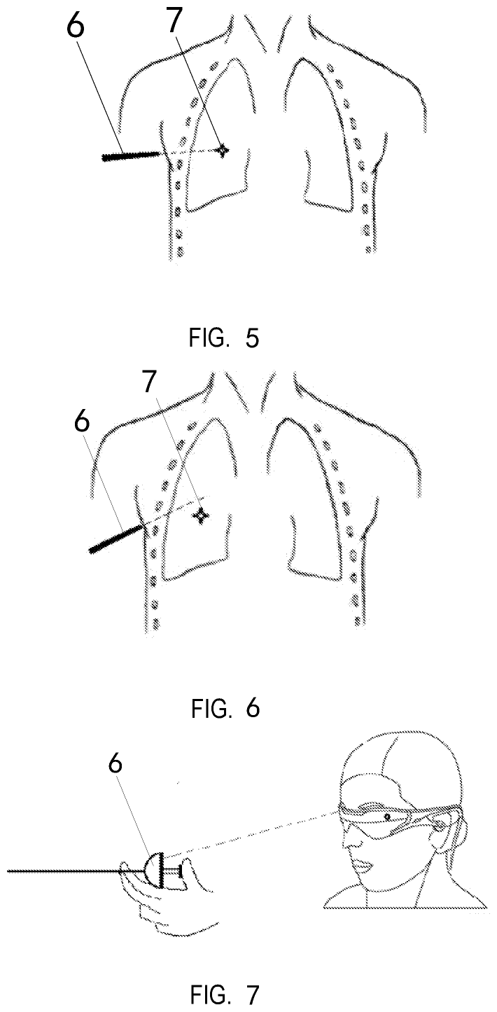

Taking the identifier being a puncture needle as an example, referring to FIG. 5, in the process of alignment, a puncture needle 6 is correctly directed to a target site 7, and at this time, the first recognition result of the surgical navigation system is that the surgical scene image contains the puncture needle, and the third recognition result is that the puncture needle is correctly directed to the target site, then an interaction instruction for “triggering distance measurement to display the distance between the puncture needle's tip and the target site” is generated. Referring to FIG. 6, in the process of alignment, the direction of the puncture needle 6 deviates from the target site 7, and at this time, the first recognition result of the surgical navigation system is that the surgical scene image contains the puncture needle, and the third recognition result is that the direction of the puncture needle deviates from the target site, then an interaction instruction for “triggering angle measurement to display prompt message” is generated.

When the operator discovers an error in the alignment of the surgical navigation system and re-alignment is required, the operator may execute a specific action on the identifier, such as changing the position of the identifier or changing the posture of the identifier (orientation and/or angle), based on which the system enters the process of re-alignment.

As an optional embodiment of the instruction obtaining module, when the instruction obtaining module is used to, based on the first recognition result, obtain the corresponding interaction instruction, it is specifically used for:
obtaining a fourth recognition result by executing image recognition on the surgical scene image, wherein the fourth recognition result is used to represent a degree to which the identifier is obscured;
based on the first recognition result and the fourth recognition result, obtaining the corresponding interaction instruction.

In this embodiment, the operator may control the surgical navigation system by obscuring the identifier to improve the convenience of control.

When the operator's hand partially obscures the two-dimensional code on the surgical device, it indicates that the final purpose of the puncture operation is being executed or has been accomplished: fluid injection or device implantation. At this time, a finishing operation process of the surgical navigation system needs to be triggered. Referring to FIG. 7, taking the identifier being the two-dimensional code configured on the puncture needle 6 as an example, in the figure, the two-dimensional code on the puncture needle 6 is partially obscured by the operator's hand, and at this time, the first recognition result of the surgical navigation system is that the surgical scene image contains the two-dimensional code, and the fourth recognition result is that the two-dimensional code is partially obscured, then an interaction instruction for “triggering a finishing operation process of the surgical navigation system” is generated, and a surgical navigation process of “finishing operation process of the surgical navigation system” is executed. Specifically, when the part of the identifier that is obscured exceeds a preset ratio value, the identifier is considered to be partially obscured. The preset ratio value may be set to be 10%, and the like.

As an optional embodiment of the instruction obtaining module, when the instruction obtaining module is used to, based on the first recognition result, obtain the corresponding interaction instruction, it is specifically used for:
obtaining a fifth recognition result by executing image recognition on the surgical scene image, wherein the fifth recognition result is used to represent an absolute motion trajectory or a relative motion trajectory of the identifier, wherein the absolute motion trajectory is a motion trajectory of the identifier relative to a stationary object, and the relative motion trajectory is a motion trajectory of the identifier relative to a set person;
based on the first recognition result and the fifth recognition result, obtaining the corresponding interaction instruction.

In the technical solution of this embodiment, the corresponding interaction instruction is automatically generated based on the motion trajectory of the identifier. Specifically, the motion trajectory may be the absolute motion trajectory or the relative motion trajectory, wherein the absolute motion trajectory is a motion trajectory relative to a stationary object, for example, a floor, an operating table; whereas, the relative motion trajectory is a motion trajectory relative to a set personnel, e.g., an operator.

Taking the identifier being a two-dimensional code proposed on a puncture needle as an example, when the operator rotates the puncture needle, the two-dimensional code moves. According to the absolute motion trajectory of the two-dimensional code, the corresponding interaction instruction is generated, for example, where the two-dimensional code is recognized to rotate for one circumference, the interaction instruction for “triggering to hid rib pattern” is generated.

When the operator discovers an error in the alignment of the surgical navigation system and re-alignment is required, the operator may execute a specific action on the identifier, such as changing the position of the identifier or changing the posture of the identifier, based on which the system enters the process of re-alignment.

As an optional embodiment of the instruction obtaining module, when the instruction obtaining module is used to, based on the first recognition result, obtain the corresponding interaction instruction, it is specifically used for:
based on at least two of the surgical navigation stage, the second recognition result, the third recognition result, the fourth recognition result and the fifth recognition result, as well as the first recognition result, obtaining the corresponding interaction instruction.

More specifically, the corresponding interaction instruction is obtained based on at least three of the surgical navigation stage, the second recognition result, the third recognition result, the fourth recognition result and the fifth recognition result, as well as the first recognition result.

More specifically, the corresponding interaction instruction is obtained based on the surgical navigation stage, the first recognition result, the second recognition result, the third recognition result, the fourth recognition result and the fifth recognition result.

Since the surgical navigation system has different requirements for navigation information at different stages, the current process is determined according to the different requirements for navigation information. A plurality of identifiers may be proposed in the surgical scene. When the camera is facing the first identifier, it means that the surgical operation is in the preparation stage or the alignment stage. When the camera is facing the second identifier, it means that it is already in the stage of puncture needle starting to enter the human body. When the puncture needle enters the human body, the surgeon needs to focus his attention to avoid too much interference information to be displayed at this time but only the most important information.

The surgical navigation system comprises a navigation information display module for displaying the corresponding surgical navigation information at the corresponding position in a real scene in a manner of augmented reality or hide the surgical navigation information, e.g., according to the interaction instruction for “triggering to hide rib pattern”, the corresponding surgical navigation information is displayed after the rib pattern is hidden.

To sum up, in the surgical navigation system of the present application, different surgical navigation steps may be triggered by recognizing the same identifier at different surgical navigation stages. For example, in the process of human body alignment, by recognizing the plane positioning board again, the current alignment process may be reset. If the puncture needle is recognized in the process of alignment, it is defined as a recognition needle serving to determine the position of a marker point on the surface of the human body, and when the puncture needle is recognized in the puncture process, a puncture navigation task is executed.

In the surgical navigation system of the present application, different surgical navigation steps may be triggered by recognizing different angles or different motion trajectories of the same identifier at the same surgical navigation stage. For example, in the process of puncture navigation, when the operator operates the puncture needle by rotating it for one turn in clockwise direction, the rib pattern is hidden so that the operator can see the surgical area behind the rib more clearly.

In the surgical navigation system of the present application, different surgical navigation steps may be triggered by recognizing different degrees to which the same identifier is obscured. For example, in the process of puncture navigation, when it occurs that the puncture needle is partially obscured by a thumb and the obscurity lasts for a certain period of time, it is considered that the action of releasing the device inside the puncture needle has been performed, and at this time, the previous position of the tip of the needle is recorded, namely a surgical record of a release point of the device for subsequent surgical analysis.

In the surgical navigation system of the present application, different surgical navigation steps may be triggered by recognizing the relative position of the same identifier in the preset space or the relative distance between the same identifier and the preset target. For example, in the process of alignment, the recognition board is placed in proximity of the alignment point at a recorded position, and then only the position information of this point is reset. The alignment efficiency can be improved.

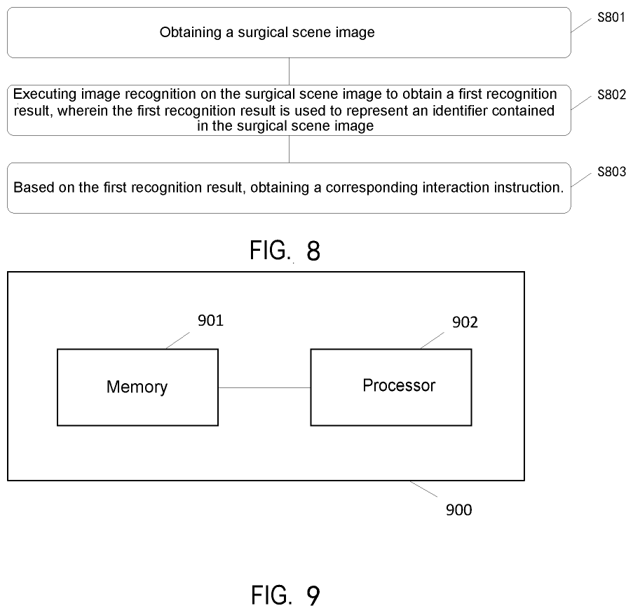

Referring to FIG. 8, an information interaction method for a surgical navigation system comprises:
S801, obtaining a surgical scene image;
S802, executing image recognition on the surgical scene image to obtain a first recognition result, wherein the first recognition result is used to represent an identifier contained in the surgical scene image;
S803, based on the first recognition result, obtaining a corresponding interaction instruction.

In the information interaction method for the surgical navigation system in the embodiment of the present application, an identifier contained in a surgical scene image captured by a camera may be automatically recognized, and a corresponding interaction instruction may be obtained based on the identifier contained in the surgical scene image. This enables the surgical navigation system executing the information interaction method of the present embodiment to obtain the corresponding interaction instruction based on the surgical scene image with the identifier captured by an operator, and the surgical navigation system can be controlled by the interaction instruction to execute a corresponding surgical navigation step without the need to operate by using voices, gestures, and the like, so that the implementation of the technical solution of the present application can reduce the chances of misjudgments when the surgical navigation system is being controlled.

Wherein, it can be known that the operator may photograph the surgical scene by a camera of a headwear device worn by him to collect the surgical scene image.

As an optional embodiment of the step S802, executing image recognition on the surgical scene image to obtain the first recognition result comprises:
extracting an image feature of the surgical scene image;
determining the first recognition result based on a similarity between the image feature of the surgical scene image and an image feature of the identifier.

As an optional embodiment of the step S803, based on the first recognition result, obtaining the corresponding interaction instruction comprises:
obtaining a surgical navigation stage at which the surgical navigation system is;
based on the first recognition result and the surgical navigation stage, obtaining the corresponding interaction instruction.

As an optional embodiment of the step S803, based on the first recognition result, obtaining the corresponding interaction instruction comprises:
obtaining a second recognition result by executing image recognition on the surgical scene image, wherein the second recognition result is used to represent a relative position of the identifier in a preset space, or represent a relative distance between the identifier and a preset target;
based on the first recognition result and the second recognition result, obtaining the corresponding interaction instruction.

As an optional embodiment of the step S803, based on the first recognition result, obtaining the corresponding interaction instruction comprises:
obtaining a third recognition result by executing image recognition on the surgical scene image, wherein the third recognition result is used to represent an orientation and/or angle of the identifier;
based on the first recognition result and the third recognition result, obtaining the corresponding interaction instruction.

As an optional embodiment of the step S803, based on the first recognition result, obtaining the corresponding interaction instruction comprises:
obtaining a fourth recognition result by executing image recognition on the surgical scene image, wherein the fourth recognition result is used to represent a degree to which the identifier is obscured;
based on the first recognition result and the fourth recognition result, obtaining the corresponding interaction instruction.

As an optional embodiment of the step S803, based on the first recognition result, obtaining the corresponding interaction instruction comprises:
obtaining a fifth recognition result by executing image recognition on the surgical scene image, wherein the fifth recognition result is used to represent an absolute motion trajectory or a relative motion trajectory of the identifier, wherein the absolute motion trajectory is a motion trajectory of the identifier relative to a stationary object, and the relative motion trajectory is a motion trajectory of the identifier relative to a set person;
based on the first recognition result and the fifth recognition result, obtaining the corresponding interaction instruction.

For specific technical solutions, principles and effects of the information interaction method of the above embodiments, the relevant technical solutions, principles and effects in the surgical navigation system described above can be referred.

Referring to FIG. 9, an electronic device 900 comprises a memory 901 and a processor 902. The memory 901 is used to store computer instructions. The computer instructions are executed by the processor 902 to implement the information interaction method of any of the embodiments of the present application.

The present application also provides a readable storage medium with computer instructions stored thereon. When the computer instructions are executed by a processor, the information interaction method of any of the embodiments of the present application is implemented.

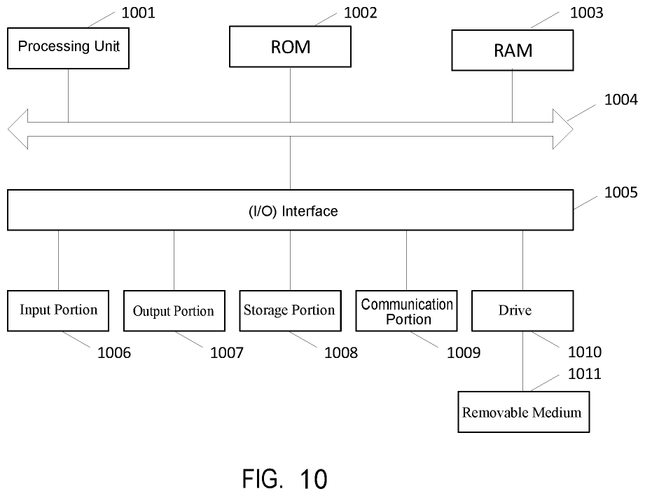

FIG. 10 is a schematic diagram of a structure of a computer system suitable for use to perform the method of one embodiment of the present application.

Referring to FIG. 10, the computer system comprises a processing unit 1001 which may execute various processes in the embodiment shown in the drawings above in accordance with programs stored in a Read-Only Memory (ROM) 1002 or programs loaded from a storage portion 1008 into a Random Access Memory (RAM) 1003. Various programs and data required for system operation are also stored in the RAM 1003. The processing unit 1001, the ROM 1002 and the RAM 1003 are connected to each other via a bus 1004. An input/output (I/O) interface 1005 is also connected to the bus 1004.

The following components are connected to the I/O interface 1005: an input portion 1006 including a keyboard, a mouse, etc.; an output portion 1007 including a Cathode Ray Tube (CRT), a Liquid Crystal Display (LCD), etc., and a speaker, etc; a storage portion 1008 including a hard disk, etc.; and a communication portion 1009 including a network interface card, such as a LAN card, a modem, etc. The communication portion 1009 executes communication processing via a network such as Internet. A drive 1010 is also connected to the I/O interface 1005 as needed. A removable medium 1011, such as a disk, a CD-ROM, a magnetic disk, a semiconductor memory, etc., is mounted on the drive 1010 as needed to allow the computer programs read from it to be installed into the storage portion 1008 as needed. Wherein, the processing unit 1001 may be implemented as a CPU, GPU, TPU, FPGA, NPU, and the like.

In particular, according to the embodiments of the present application, the method described above may be implemented as a computer software program. For example, the embodiments of the present application include a computer program product comprising a computer program tangibly contained on a readable medium thereof. The computer program comprises program codes for executing the method in the drawings. In such an embodiment, the computer program may be downloaded and installed from a network via the communication portion 1009 and/or installed from the removable medium 1011.

In the description of this specification, the reference terms “an embodiment/manner”, “some embodiments/manners”, “example”, “specific example”, or “some examples”, and the like mean that the specific features, structures, materials, or characteristics described in conjunction with the embodiments/manners or examples are included in at least one embodiment/manner or example of the present application. In this specification, the schematic expressions of the above terms do not have to be directed to the same embodiment/manner or example. Moreover, the specific features, structures, materials, or characteristics described may be combined in a suitable manner in any one or more of the embodiments/manners or examples. Furthermore, without contradicting each other, those skilled in the art may combine and associate different the embodiments/manners or examples and features of different embodiments/manners or examples described in this description.

Furthermore, the terms “first” and “second” are used for descriptive purposes only and cannot be understood as indicating or implying relative importance or implicitly specifying the number of technical features indicated. Thus, features defined with the terms “first”, “second” may expressly or impliedly include at least one of such features. In the Description of the present application, “a plurality of” means at least two, e.g., two, three, and the like, unless otherwise expressly and specifically defined.

It should be understood by those skilled in the art that the above embodiments are merely for the purpose of clearly illustrating the present disclosure and are not intended to limit the scope of the present disclosure. For those skilled in the art, other changes or variations may be made on the basis of the above disclosure, and such changes or variations remain within the scope of the present disclosure.

1. A surgical navigation system, characterized in, comprising:
an image obtaining module, for obtaining a surgical scene image;
an image recognition module, for executing image recognition on the surgical scene image to obtain a first recognition result, wherein the first recognition result is used to represent an identifier contained in the surgical scene image;
an instruction obtaining module, for, based on the first identification result, obtaining a corresponding interaction instruction;
an instruction execution module, for, based on the interaction instruction, controlling the surgical navigation system to execute a corresponding surgical navigation step.

2. The surgical navigation system according to claim 1, characterized in that when the image recognition module is used for executing the image recognition on the surgical scene image to obtain the first recognition result, it is specifically used for:
extracting an image feature of the surgical scene image;
determining the first recognition result based on a similarity between the image feature of the surgical scene image and an image feature of the identifier.

3. The surgical navigation system according to claim 1, characterized in that when the instruction obtaining module is used for, based on the first recognition result, obtaining the corresponding interaction instruction, it is specifically used for:
obtaining a surgical navigation stage at which the surgical navigation system is;
based on the first recognition result and the surgical navigation stage, obtaining the corresponding interaction instruction.

4. The surgical navigation system according to claim 1, characterized in that when the instruction obtaining module is used for, based on the first recognition result, obtaining the corresponding interaction instruction, it is specifically used for:
obtaining a second recognition result by executing image recognition on the surgical scene image, wherein the second recognition result is used to represent a relative position of the identifier in a preset space, or represent a relative distance between the identifier and a preset target;
based on the first recognition result and the second recognition result, obtaining the corresponding interaction instruction.

5. The surgical navigation system according to claim 1, characterized in that when the instruction obtaining module is used for, based on the first recognition result, obtaining the corresponding interaction instruction, it is specifically used for:
obtaining a third recognition result by executing image recognition on the surgical scene image, wherein the third recognition result is used to represent an orientation and/or angle of the identifier;
based on the first recognition result and the third recognition result, obtaining the corresponding interaction instruction.

6. The surgical navigation system according to claim 1, characterized in that when the instruction obtaining module is used for, based on the first recognition result, obtaining the corresponding interaction instruction, it is specifically used for:
obtaining a fourth recognition result by executing image recognition on the surgical scene image, wherein the fourth recognition result is used to represent a degree to which the identifier is obscured;
based on the first recognition result and the fourth recognition result, obtaining the corresponding interaction instruction.

7. The surgical navigation system according to claim 1, characterized in that when the instruction obtaining module is used for, based on the first recognition result, obtaining the corresponding interaction instruction, it is specifically used for:
obtaining a fifth recognition result by executing image recognition on the surgical scene image, wherein the fifth recognition result is used to represent an absolute motion trajectory or a relative motion trajectory of the identifier, wherein the absolute motion trajectory is a motion trajectory of the identifier relative to a stationary object, and the relative motion trajectory is a motion trajectory of the identifier relative to a set person;
based on the first recognition result and the fifth recognition result, obtaining the corresponding interaction instruction.

8. An information interaction method for a surgical navigation system, characterized in, comprising:
obtaining a surgical scene image;
executing image recognition on the surgical scene image to obtain a first recognition result, wherein the first recognition result is used to represent an identifier contained in the surgical scene image obtained by the recognition;
based on the first recognition result, obtaining a corresponding interaction instruction.

9. An electronic device, comprising a memory and a processor, the memory used to store computer instructions, characterized in that the computer instructions are executed by the processor to implement the method according to claim 8.

10. A readable storage medium with the computer instructions stored thereon characterized in that when the computer instructions are executed by a processor, the method according to claim 8 is implemented.
